(19) United States
(12) Patent Application Publication
(10) Pub. No.: US 2019/0111235 A1
(43) Pub. Date: Apr. 18, 2019

(54) CATHETER HUB WITH REMOVABLE EXTENSIONS

(71) Applicant: Avasys, LLC, Columbia, MD (US)

(72) Inventor: Cameron C. Jones, Pikesville, MD (US)

(21) Appl. No.: 16/096,518

(22) PCT Filed: Apr. 26, 2017
(86) PCT No.: PCT/US17/29610

§ 371 Date: Oct. 25, 2018

Related U.S. Application Data
(60) Provisional application No. 62/328,191, filed on Apr. 27, 2016.

(51) Int. Cl. A61M 25/00; A61M 39/28; A61M 39/10; A61M 39/22

(52) U.S. Cl. CPC A61M 25/0097; A61M 39/284; A61M 25/0026; A61M 39/223; A61M 39/105

(57) ABSTRACT

A multi-lumen catheter hub with a plurality of attachment members is provided. The catheter hub allows removal of the external fluid connections of an elongated percutaneous medical article, such as a catheter or cannula. The percutaneous medical article suitably contains a clamping mechanism that prevents fluid movement within the intraluminal space of the percutaneous medical article when the fluid connections are removed. The attachment members are single-use, disposable components that provide intraluminal access for a variety of medical articles and are removed following clinical use. Retaining the catheter transition allows the catheter to be secured using methods common to the art.

### RELATED APPLICATIONS

This application claims the benefit of priority under 35 U.S.C. § 119(e) to U.S. Provisional Application No: 62/328,191 filed Apr. 27, 2016, which is incorporated herein by reference in its entirety.

### FIELD OF INVENTION

The present invention relates generally to medical devices. More particularly, the present invention relates to a connecting hub for use with a catheter.

### BACKGROUND

Many patients with chronic diseases or who are critically ill require frequent administration of fluids for nutritional or medicinal purposes. These medications are oftentimes delivered through an intravenous catheter such as a central venous catheter (CVC), peripherally inserted central line (PICC), and midline catheter, which provides vascular access and may be kept in place for durations lasting several days up to several months. For example, patients requiring dialysis may visit a healthcare setting for a few hours each week or over a particular rotation, but outside of these dialysis sessions, the catheter is unused while remaining indwelling to the patient for future visits.

Modern medical catheters that have a portion of the catheter body extending outside the patient (“percutaneous”) consist of an indwelling portion and an external portion that primarily acts as a conduit to the indwelling portion. Many catheters are multi-luminal, where each lumen may serve different functions depending on anatomic location and/or prescribed clinical need. External to the patient, the multi-lumen catheter bifurcates into single lumen lines, where the distal ends of the lines have a standard medical fitting (e.g., luer) for connecting infusion lines or various medical equipment, and a clamp to prevent fluid movement and air embolism when the catheter is not being accessed. The site of bifurcation is often called the “hub” or “transition” and is traditionally a molded region that connects the indwelling catheter to the external extension(s), and is disposed adjacent to the insertion site. The intravascular portion of the catheter is usually rich in biocompatibility, and may consist of antimicrobial features and may be optimized for fluid dynamic needs, but the hub and extensions, lying external to the patient, generally do not have as rigorous demands on biocompatibility and fluid dynamic function, acting primarily as a conduit for the passage of fluids.

Following placement of the intravascular catheter, it is often necessary to secure the catheter to the patient when used for extended periods of time to prevent axial displacement of the catheter with regards to the anatomical position thereof. Securement of the catheter is generally accomplished by one of three means: suturing the catheter hub to the patient's skin through eyelets in the wings extending from the molded hub; applying an adhesive over the catheter hub thereby securing the hub to the patient's skin; or placing the hub in a semi-flexible securement device which is held to the patient's skin by an adhesive base. The securement methods minimize axial movement of the catheter by limiting displacement due to tugging of external extensions with environmental articles.

Medical catheters are manufactured using polymeric compounds such as silicone, polyethylene, polyurethane, and polytetrafluorethylene to increase biocompatibility and longevity of use. Despite precautions, catheter-related infections are a frequent and growing concern, having significant consequences to patient morbidity and mortality, and greatly taxing to healthcare resources. Infections stem from bacterial adsorption on the catheter surface, giving way to a prolific growth of a highly antibiotic-resistant community of cells called biofilm. The predominant sources of bacteria that colonize on intravascular catheters arise external to the patient, either through contamination of the intraluminal surface due to non-sterile catheter access (i.e., incomplete disinfection prior to line access) or along the extraluminal surface from microbial egress through the insertion site, facilitated by pistoning (back-and-forth movement) of the catheter. Every time these central lines are handled pose a potential risk for contamination.

The long-hanging catheter extensions can cause abrasions to patient skin, are uncomfortable, and may become tangled in clothing or other articles that cause minute axial movement, where such movements can introduce pathogens along the extraluminal surface through the skin insertion site. Caps or needleless connectors which often terminate the distal end of a catheter must be disinfected prior to line access and may be removed and/or reattached without proper cleaning. These connectors may also contain regions that are difficult to disinfect, harboring bacteria that may subsequently be introduced into the patient's bloodstream with the next infusion. Finally, the catheter extensions and accessories thereof (caps, clamps, etc.) can sometimes break, become unsnapped, or kink, which can compromise the entire catheter and present a major risk in patient safety.

It would therefore be advantageous to provide a connecting hub with close proximity to the catheter insertion site that allows attachment and removal of various extensions, reduces the total surface area of the extravascular portion and the predominant site of a contaminant's origin, facilitates improved placement of insertion-site dressing, and substantially reduces the potential for snagging, irritation, and other problems associated with dangling catheter extensions.

### SUMMARY

The present invention provides a catheter capable of achieving the above-described advantages.

In one aspect, a catheter assembly is provided that comprises a hub having an outer housing defining a proximal end and a distal end. The distal end of the outer housing defines an opening into an inner space defined by the outer housing, and the proximal end of the housing defines a proximal connector for coupling to an intravenous catheter tube which includes a single lumen or multiple lumens. Additionally, disposed between the distal and proximal ends is an occluding assembly that blocks a section of a fluid channel The catheter also includes an attachment member that connects to the distal end of the hub to seal the catheter while not in use. The attachment member specifically connects to a connector region of the hub and is secured by a locking member that engages with a protrusion formed on the hub.

In an additional aspect, the occluding assembly is a clamping mechanism that includes a clamping member that engages with a clamping support and the clamping support engages with a section of the hub to maintain a position within the hub.

In another aspect, the catheter further includes an extension tube assembly that is releasably attached to the connector. In particular, the extension tube assembly can be released from the catheter while the catheter unit is affixed to a patient. The extension tube assembly may be releasably engaged to the catheter unit by a mechanical system and may further include a locking cap where an extension tube attaches to the hub. Further, the extension tube comprises a number of extension tubes in a range of 1-4 extension tubes that converge at the locking cap to connect to the hub.

Accordingly to another aspect of the present invention, a catheter assembly is provided that comprises a hub having an outer housing defining an opening into an inner space defined by the outer housing, and the proximal end of the housing defines a proximal connector for coupling to an intravenous catheter tube. Additionally, the catheter includes an occluding assembly that blocks a section of a fluid channel and is disposed between the distal and proximal ends of the hub. An attachment member connects to the distal end of the hub to form a fluid-tight seal.

In an additional aspect, the attachment member includes an attachment cap in which fluid connectors are formed and a deformable insert that is fitted into the attachment cap. The insert includes openings that fill a gap between the fluid connectors and a fluid connector region at the distal end of the hub to form the fluid-tight seal.

In another aspect, the attachment member includes a female luer connector and a slider that diverts fluid through lumens of the catheter. The slide includes an indicator that provides an indication of a position of the slider. Further, the slider moves laterally within a channel of the attachment member and includes multiple openings and closures. A fluid lumen proximal to the female luer connector bifurcates into multiple fluid channels.

In a further aspect, the attachment member includes a female luer connector and a valve assembly that has a portion extending from the attachment member to allow the fluid to be directed to a particular lumen. The valve assembly is rotated to restrict and permit flow into multiple lumens.

According to yet another aspect of the present invention, a method is provided for releasably attaching a catheter assembly. The method includes affixing a catheter having a hub and occluding assembly as described above to a surface of a patient and then depressing the occluding assembly to block fluid movement. Additionally, the extension tube assembly can be removed from the catheter that is affixed to the patient surface. The method also includes attaching the attachment member as described above to the distal end of the hub to seal the catheter. The extension tube assembly can then be re-attached to the catheter during treatment of the patient. The catheter is affixed to the patient surface by suturing or using an adhesive.

Notably, the present invention is not limited to the combination of catheter elements as listed above and may be assembled in any combination of the above-described elements. Moreover, references to multi-lumen devices are intended to be inclusive of single lumen catheters as well.

Other aspects of the invention are disclosed infra.

### BRIEF DESCRIPTION OF THE DRAWINGS

The accompanying drawings provide visual representations, which will be used to more fully describe the representative embodiments disclosed herein and can be used by those skilled in the art to better understand them and their inherent advantages. In these drawings, like reference numerals identify corresponding elements and:

FIG. 1 illustrates an exploded view of a catheter connector and an attaching member according to an exemplary embodiment of the present invention;

FIG. 2 illustrates a cross-sectional view of the catheter connector according to an exemplary embodiment of the present invention;

FIG. 3 illustrates an exploded view of the assembly of the clamping mechanism with the catheter hub according to an exemplary embodiment of the present invention;

FIGS. 4A-5B illustrate partial cross-sectional views of the clamping mechanism on a particular region disposed within the proximal and distal ends of the catheter connector according to the exemplary embodiment of the present invention;

FIG. 5 illustrates a perspective view of an attachment member with locking features which engage with a matching region of the catheter hub according to an exemplary embodiment of the present invention;

FIG. 6 illustrates an attachable extension tube assembly with various components common to traditional vascular catheters used in conjunction with the catheter hub according to an exemplary embodiment of the present invention;

FIG. 7 illustrates an exploded view of an exemplary attachment member having an elastic insert which deforms around fluid connectors to establish a fluid-tight seal according to an exemplary embodiment of the present invention;

FIG. 8 illustrates a perspective view of an attachment member used with a single syringe and in conjunction with the catheter hub according to an exemplary embodiment of the present invention;

FIGS. 9A-9B illustrate internal features of an attachment member used with a single syringe and in conjunction with the catheter hub according to an exemplary embodiment of the present invention;

FIGS. 10A-10C illustrate an attachment member with a valve mechanism used in conjunction with the catheter hub according to an exemplary embodiment of the present invention; and

FIG. 11 illustrates a perspective view of an attachment member with fixed bifurcation for connecting medical articles and used in conjunction with the catheter hub according to an exemplary embodiment of the present invention.

### DETAILED DESCRIPTION

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items.

Unless specifically stated or obvious from context, as used herein, the term “about” is understood as within a range of normal tolerance in the art, for example within 2 standard deviations of the mean. “About” can be understood as within 10%, 9%, 8%, 7%, 6%, 5%, 4%, 3%, 2%, 1%, 0.5%, 0.1%, 0.05%, or 0.01% of the stated value. Unless otherwise clear from the context, all numerical values provided herein are modified by the term “about.”

The presently disclosed subject matter will now be described more fully hereinafter with reference to the accompanying drawings, in which some, but not all embodiments of the inventions are shown. Like numbers refer to like elements throughout. The presently disclosed subject matter may be embodied in many different forms and should not be construed as limited to the embodiments set forth herein; rather, these embodiments are provided so that this disclosure will satisfy applicable legal requirements. Indeed, many modifications and other embodiments of the presently disclosed subject matter set forth herein will come to mind to one skilled in the art to which the presently disclosed subject matter pertains, having the benefit of the teachings presented in the foregoing descriptions and the associated drawings. Therefore, it is to be understood that the presently disclosed subject matter is not to be limited to the specific embodiments disclosed and that modifications and other embodiments are intended to be included within the scope of the appended claims.

The present invention is directed to a multi-lumen catheter with a plurality of attachment members (e.g., members that are attachable to the catheter assembly). More particularly, the present invention is directed to a technology that allows for the removal of the external fluid connections of an elongated percutaneous medical article, such as a catheter or cannula. The hub of the catheter contains a clamping mechanism (e.g., an occluding assembly) disposed therein that allows attachment and detachment of various attachable members. Retaining the catheter transition (hub) allows the catheter to be secured using methods common to the art.

For the present invention, a catheter with removable extensions having been initially attached and resembling a common percutaneous catheter is inserted into a patient by one skilled in the art. Following clinical technique known by one of ordinary skill in the art, the catheter is checked for patency, flushed, primed with a fluid solution, and secured to the patient's skin through a preferred securement method. When the catheter is not required for immediate clinical use, the extensions may be removed and a cap may be attached thereto. Upon removal of the extension set, the extravascular portion of the catheter has a lower profile and is less prone to entanglement with environmental articles.

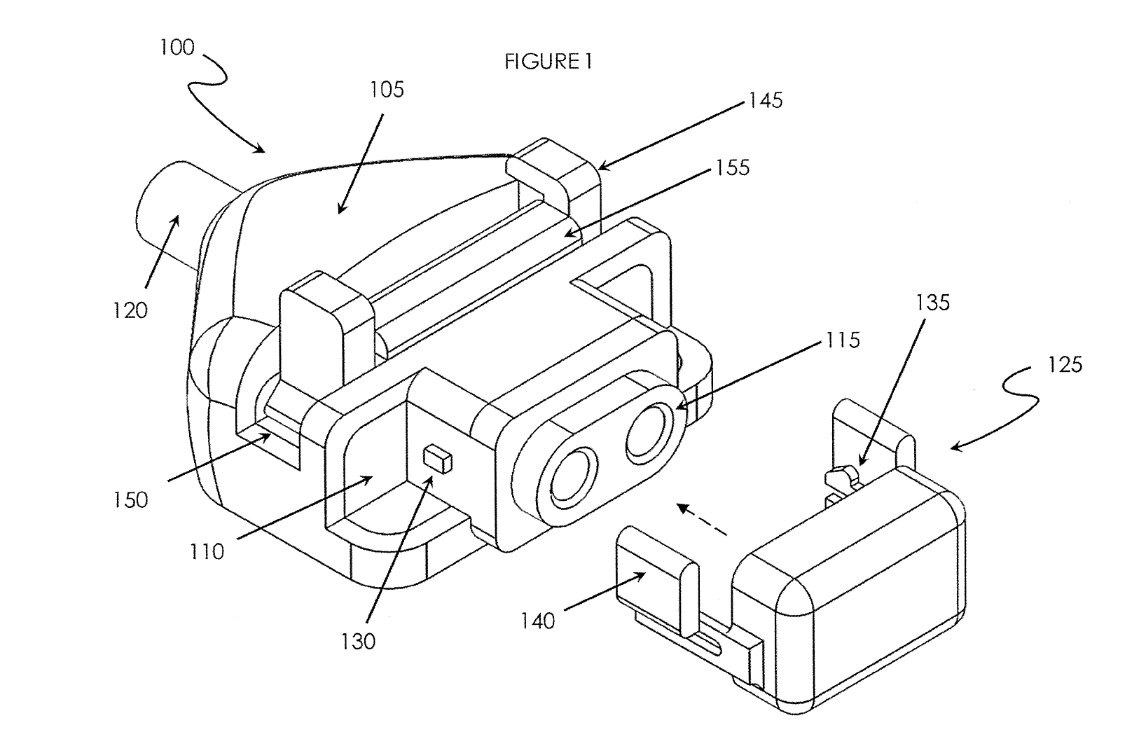

FIG. 1 illustrates an exploded view of the distal catheter hub assembly and attachment member according to an exemplary embodiment of the present invention. As illustrated in FIG. 1, a distal catheter hub 100 may include a moldable member 105 that joins a rigid plastic unit 110 to the indwelling catheter 120. The rigid member 110 contains several features that enable connecting various parts and assemblies. In particular, the rigid member 110 comprises a fluid connector region 115 in which mating fluid connectors attach and allow fluid access to the lumen(s) of the indwelling catheter. The rigid member 110 also contains a moldable feature such as a boss or recess 130 (e.g., a protrusion, geometric feature, etc.) which engage with an attachment member and securely hold the members together.

As noted with respect to FIG. 1, the catheter hub 100 may also contains a member or assembly disposed between the proximal and distal ends of the catheter hub 100 that provides a method for occluding the fluid lumen(s). In the figure illustration, the clamping member 155 engages with a clamping support 145 to clamp a section of the fluid channel(s) disposed between the proximal and distal ends of the catheter hub 100. The clamping support 145 engages with a section of the catheter hub 150 to maintain its position within the catheter hub 100.

Further, with respect to FIG. 1, the present invention includes an attachment member 125 that can take on several forms and functions; several of exemplary embodiments of the attachment member 125 are presented in subsequent figures, and as stated previously, these embodiments provide only a sampling of the many different forms and functions these attachment members may be derived. In particular, the attachment member 125 includes a locking member 135 that engages with the protrusion 130 formed on the catheter hub 100. In the present embodiment, the locking mechanism 135 has an extended feature 140 to assist personnel in disengaging the locking mechanism 135, which may be concealed or difficult to detach with fingers or in the absence of any particular tool.

Also with respect to FIG. 1, the attachment and detachment of the attachment member 125 is preceded by depressing the clamping member 155 to prevent fluid movement upon removal of the attachment member. According to a preferred embodiment of the present invention, the catheter hub does not contain any surface that must be disinfected prior to connecting medical articles since the inner surface(s) of the disposable attachable members are presumed sterile. Nevertheless, the fluid connector region 115 of the catheter hub contains no obscured surfaces such that every face of the catheter hub may be disinfected should the caretaker deem it necessary.

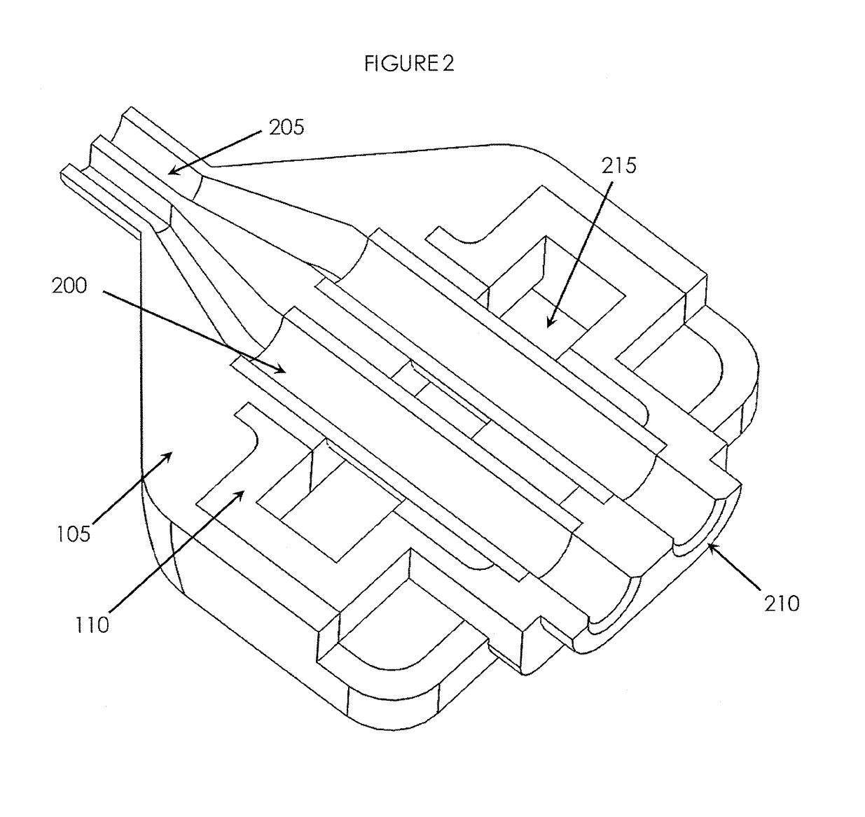

FIG. 2 illustrates a cross-sectional view of the catheter hub 100 in FIG. 1. In the present embodiment, elastic tubing 200 may be disposed between the distal and proximal ends of the catheter hub. During the manufacturing of the catheter, the elongated multi-lumen catheter is molded to the rigid member 110 with elastic tube(s) comprising a portion of the fluid channel. Bifurcation of the multi-lumen catheter occurs within the catheter hub where the individual fluid lumens 205 are molded to individual channels within the rigid member 110 comprising of elastic tubing 200 using methods common to the art such as an overmold 105, whereby the overmold 105 forms a fluid-tight junction between each (all) transition(s) in the fluid conduit. The overmold may include a plurality of openings in addition to common securement features such as wings and eyelets (not illustrated) which are attached to the patient by securement methods known to those of skill in the art.

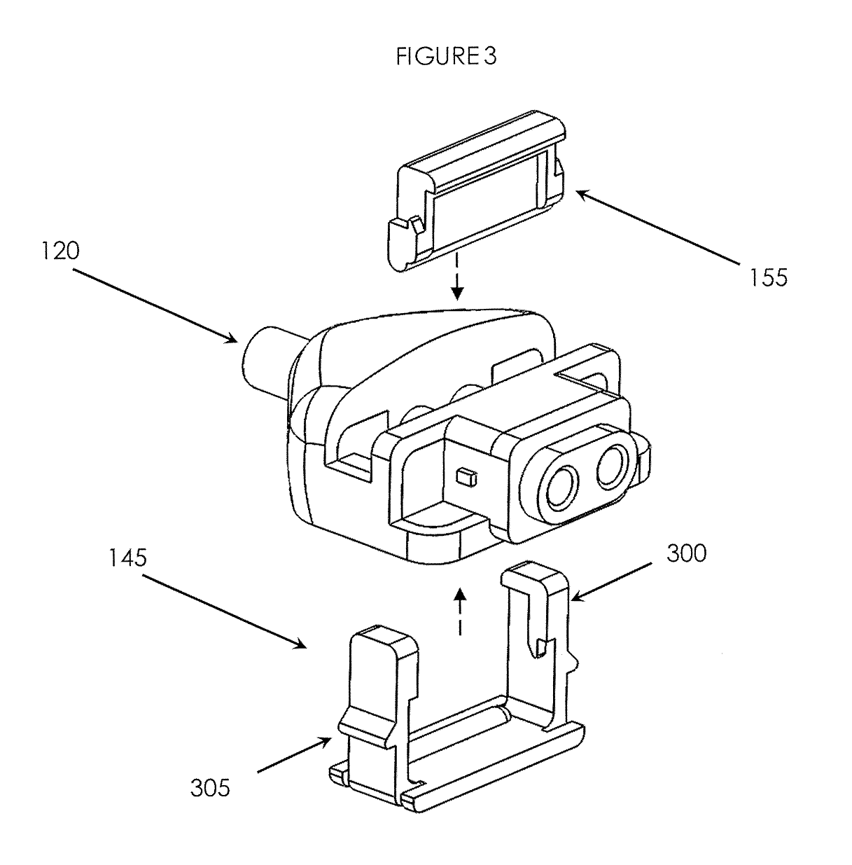

Further, with respect to FIG. 2, the rigid unit 110 contains an opening not encased by the overmold 105 that exposes the inner elastic tubing 200. In a preferred embodiment, the opening allows the placement of a clamping mechanism (see FIG. 3). The aforementioned clamping assembly may be replaced in the event of breakage (i.e., a plastic tab breaks) or removed to permit cleaning of the opening 215 should the region become soiled. Since the overmold 105 forms a sealed junction between the various components of the catheter hub, the opening 215 prevents any risk to contamination, breach, or leakage of any components of the catheter hub.

FIG. 3 shows an exploded view of the catheter hub and the attachment of a replaceable clamping mechanism between the proximal and distal ends of the catheter hub. The clamping support 145 is attached first, whereby the upright arm 300 is flexed slightly to fit within the opening of the catheter hub. Once placed within the catheter hub, the catheter support 145 is secured with a locking member 305, such as a tab. The clamping member 155 completes the assembly. In the event the part(s) require replacement, the attachment method is reversed.

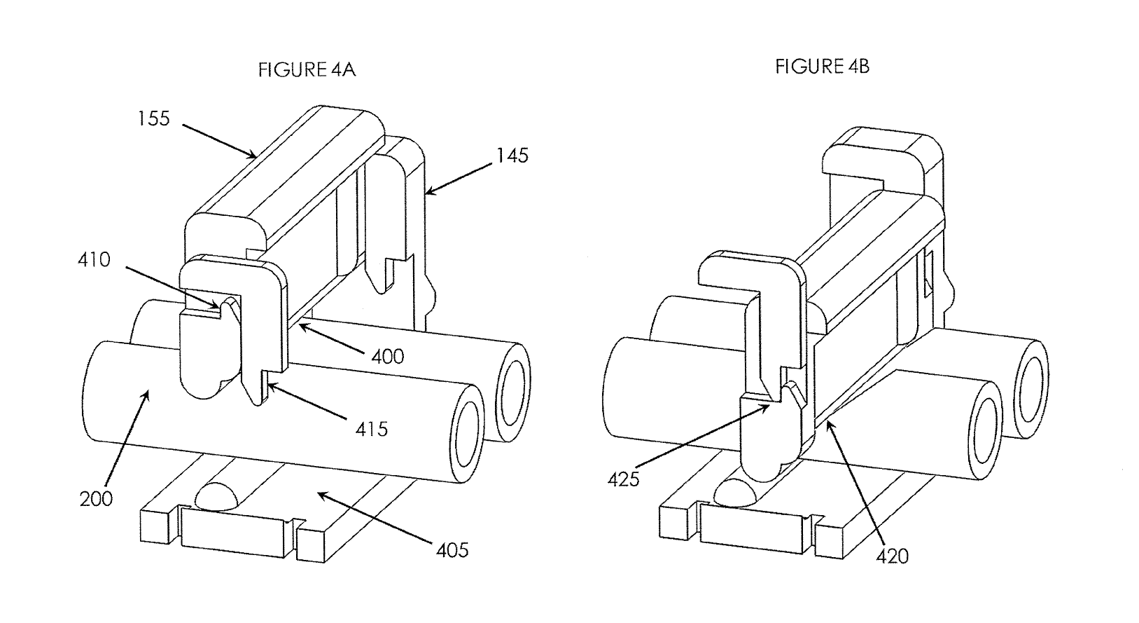

FIGS. 4A-4B illustrate a partial cross-section of a preferred embodiment of the clamping mechanism. In FIG. 4A, the elastic tubing 200 is unclamped and disposed between the top surface 405 of the bottom of the clamping support 145 and bottom edge 400 of the clamping member 155. The clamping member 155 has a locking tab 410 that engages with a region of the clamping support 415 to maintain the clamping function. In FIG. 4B, the bottom edge 420 of the clamping member is occluding one or more elastic tubing(s). The figure further illustrates the locking engagement 425 between the two clamping components.

In particular, the design of the clamping member 155 is to enable a one-handed clamping and unclamping action. In a preferred embodiment, pressing down on the top surface of the clamping member 155 engages a region of the clamping support 415, and likewise by pressing down on the top surface of the clamping member 155 followed with a slight tilt (or toggle) of the clamping member 155 disengages a region of the clamping support 415. The elastic tubing 200 provides sufficient integrity to move the clamping member 155 back to an unclamped position. The clamping member 155 may have other geometrical designs, such as a wider top surface to fully cover the opening surrounding the clamping region (see 215 of FIG. 2) or may be combined with a semi-flexible plastic shield (not shown) that provides a similar covering purpose of the opening 215 in FIG. 2.

The clamping assembly in FIGS. 4A and 4B is illustrated by two distinct members that engage with one another to accomplish an occlusive purpose of the catheter hub. However, parts may easily be manufactured that accomplish this function with a single member. Furthermore, references to a clamping action are intended to be more broadly defined as a blocking action which may be achieved by either clamping or occlusive methods. Additionally, references to elastic tubing are intended to be more broadly defined as a non-rigid material either existing as distinct members (e.g., individual tubes) or a single member with individual lumens. Therefore, the blocking action conveyed by the present invention may include the spectrum of approaches ranging from the illustrations provided herein to a single elastic member with depressible region(s) that, when depressed, form a fluid-tight obstruction, and which are disposed between the proximal and distal ends of the catheter hub.

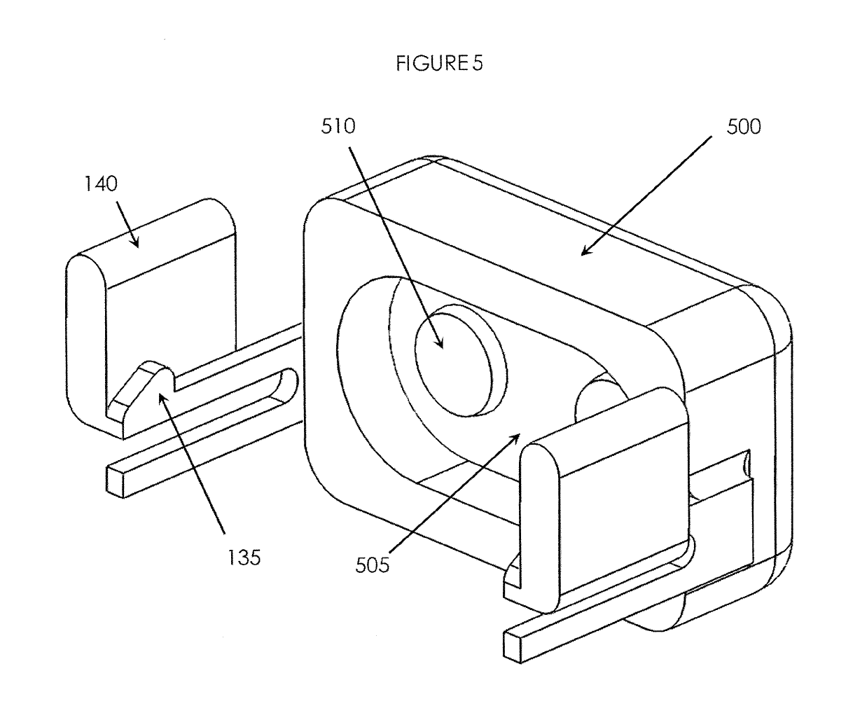

FIGS. 5 through 11 describe various adaptations of attachment members. For those skilled in the art, there are many additional forms that the attachment members may take, including forms which permit clinical functions not specifically listed in this present invention, but are apparent from the detailed descriptions of the features and advantages of the invention and as it is intended by the appended claims. The attachment members are intended to disposable components.

Particularly, FIG. 5 illustrates a terminal end cap 500 attachment. The rigid end cap 500 includes a backing 505 that seals the distal fluid openings of the catheter hub. In one embodiment, the backing 505 is formed as an elastic material with elastic protrusions 510 which press firmly against the distal surface of the catheter hub and fluid openings, creating a form-fitting fluid-tight seal.

The attachment member in FIG. 5 may be employed when the catheter is not being accessed and the catheter extensions have been removed. Therefore, the advantage of this particular attachment member is a low profile end cap which safely seals the catheter during extended non-use of the indwelling catheter (hours or days). While the present embodiment includes extensions 140 to assist with disengaging the locking member 135, there may be circumstances where patients in the community setting would benefit from removal of the extensions 140 since there would not be a need to remove the attachment member with any anticipated urgency or risk accidental disengagement of the locking members 135.

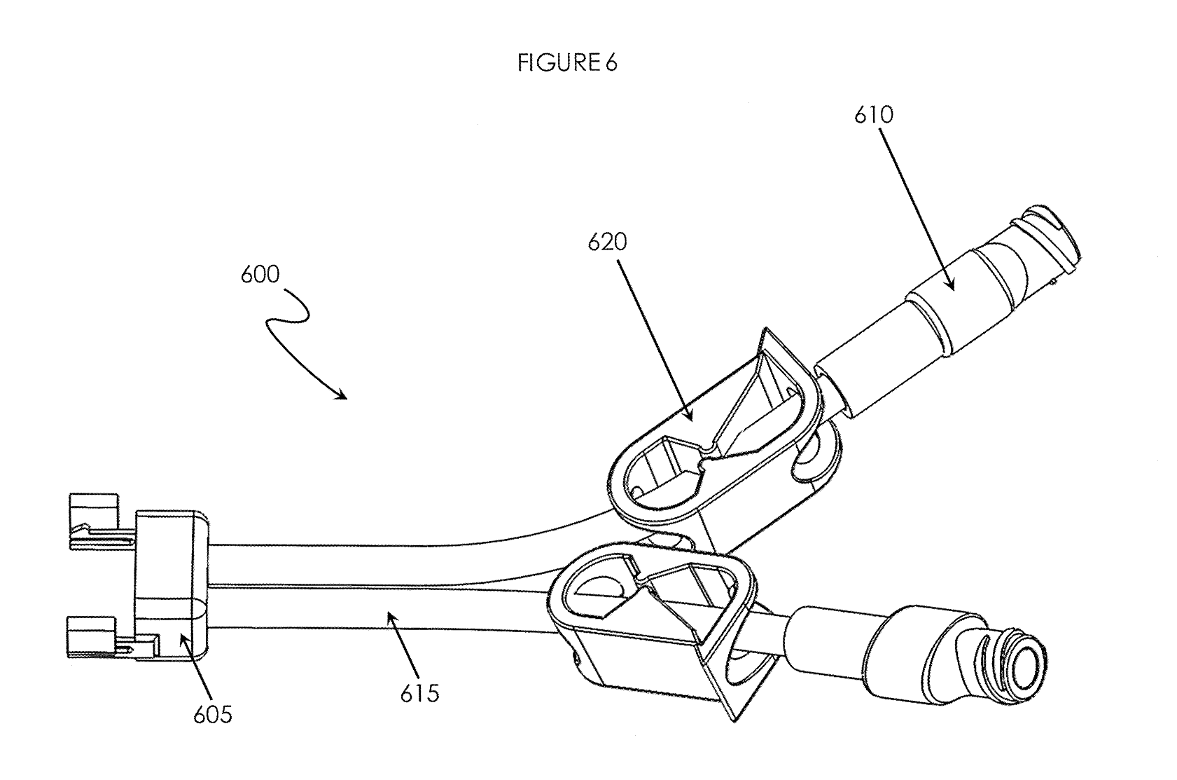

FIG. 6 illustrates an extension tube assembly 600 used in conjunction with the catheter hub according to an embodiment of the present invention, and containing various members common to the distal portion of traditional percutaneous catheters, including: extension tubing 615, clamp(s) 620, and luer fitting(s) 610. The extension tube assembly 600 includes a locking cap 605 where the single lumen extension tube(s) 615 converge to a common connector for attaching to the catheter hub. The number of extension tubes corresponds to the number of lumens of the indwelling catheter, ranging from 1-4 tubes.

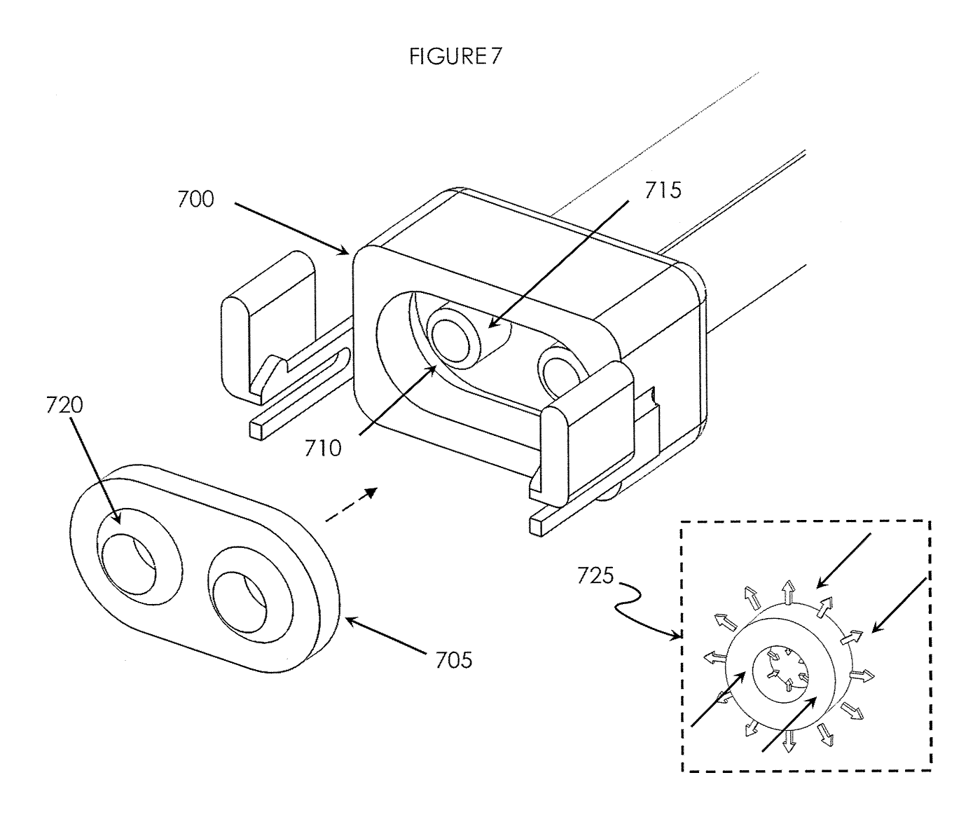

FIG. 7 shows an exploded view of an exemplary attachment member in which an elastic insert 705 rests within an open channel 710 between the attachment cap 700 and the rigid fluid connectors 715. The elastic insert 705 may be a single, flexible, synthetic polymer having high elasticity and a preformed shape, including one or more openings which fit tightly over the outer surface of the fluid connectors 715 and with a thickness slightly greater than the gap which is disposed between the catheter hub and attachment member in the absence of the elastic insert. Therefore, one or more annular regions 720 of the elastic insert 705 compress to fill gaps between the rigid fluid fittings 715 and the matching fluid connectors of the distal catheter hub.

Further with respect to the elastic insert of FIG. 7, the inlaid box 725 illustrates the displacement of the elastic insert when subjected to compressive forces (solid black arrows along the axial direction). In this example, the black arrows represent the compressive force that arises from the sandwiching of elastic insert between the catheter hub and the attachment member upon member connection. The compressive forces cause the annular elastic insert to locally bulge both externally and internally (white radial block arrows). According to a preferred embodiment of the present invention, the elastic insert will deform to fill gaps existing between the fluid connector of the attachment member 715 and the corresponding connector of the catheter hub. The elastic insert therefore ensures fluid-tight fitting between one or more fluid connectors. The advantage of the shape-adjusting (elastic) insert is evident when two or more fluid connectors are required. Since traditional medical connectors rely on a tapered surface (i.e., luer), the seal between two rigid bodies with two or more tapered fittings is likely not to be fluid-tight since both tapered surfaces are not guaranteed to be completely flush. According to an embodiment of the present invention, gaps arising from non-absolute mating surfaces are filled by the deforming elastic body, thus ensuring the fluid-tight seal.

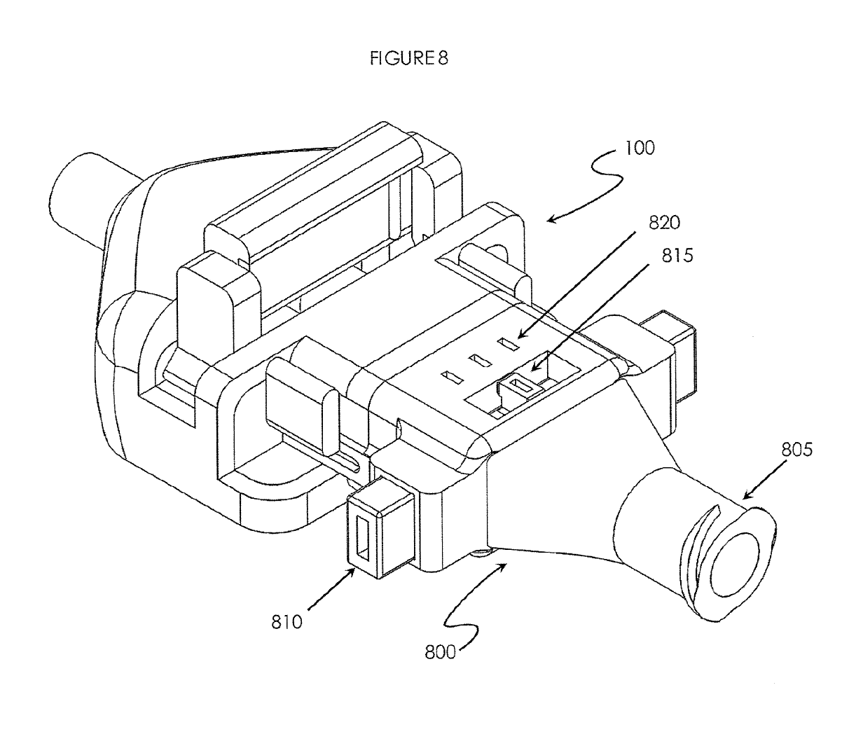

FIG. 8 illustrates a perspective view of an attachment member for use with a single syringe. The attachment member 800 contains a single female luer connector 805 and includes a slider 810 that diverts fluid through designated lumens of the multi-lumen catheter. In an embodiment of the present invention, the slider includes an indicator 815 that moves along with the position of the slider 810 and references with markings 820 on the attachment member 800 as to the function of the slider's position.

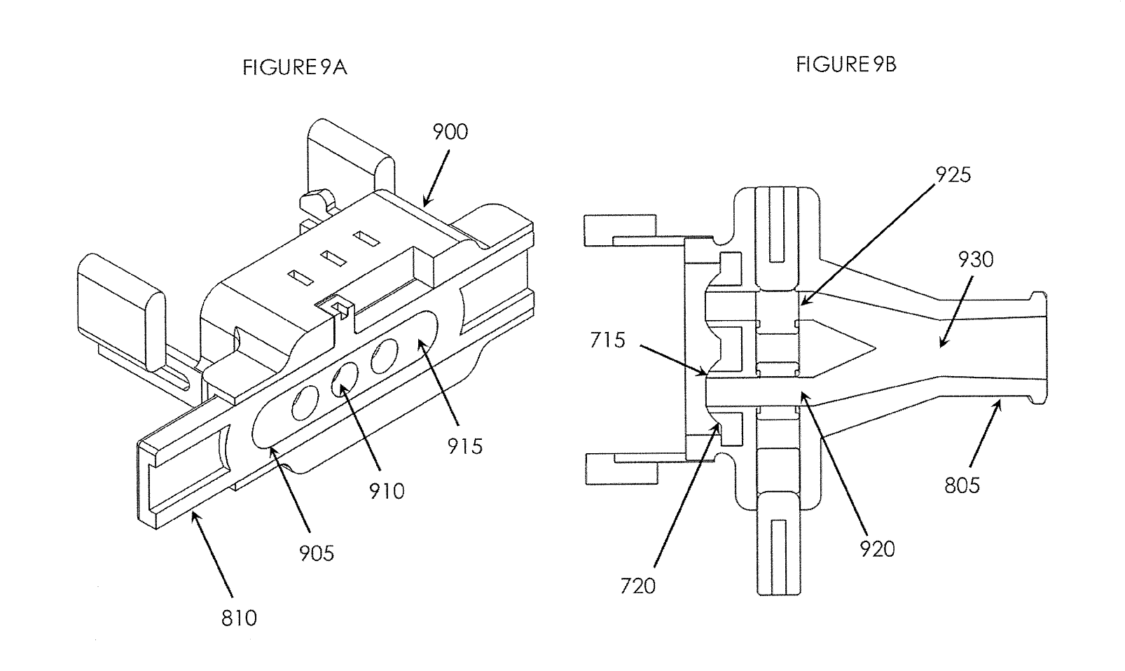

FIGS. 9A-9B illustrate cross-sectional views of the attachment member 800 in FIG. 8 according to an embodiment of the present invention. FIG. 9A is a perspective cross-sectional view that shows a slider 810 which moves laterally within a channel of the rigid attachment member 900. The slider 810 includes an insert 905 disposed within an opening of the slider 810. In a preferred embodiment of the present invention, the insert 905 is an elastic material that forms a fluid-tight seal around fluid channels using the elastic principles characterized in FIG. 7. However, the present invention is not limited thereto, and the insert may also be rigid and the two parts may be combined into one part, whereby the slider itself contains a series of fixed openings and closures. Further, the insert 905 may include one or more openings 910 and one or more blocking surfaces 915.

FIG. 9B shows an overhead cross-section of the attachment member to illustrate the fluid channels within the attachment member. The single fluid lumen 930 of the proximal luer connector 805 bifurcates into two or more fluid channels according to the number of lumens of the indwelling catheter. The arrangement of the insert opening(s) 910 and closure(s) 915 determine which fluid lumen(s) are open. According to the embodiment of the present invention, the slider 810 aligned to the left (as shown) provides fluid access to the left lumen 920 but seals the right lumen 925. According to the same embodiment of the present invention, the slider 810 moved to the center position provides fluid access to both left 920 and right 925 lumens (position not illustrated). Finally, according to the same embodiment of the present invention, the slider 810 moved to the right-most position provides fluid access to the right lumen 925, but restricts or blocks access to the left lumen 920 (position not illustrated). The advantage of the selective lumen access with a single connection site permits the caretaker to flush and/or aspirate individual lumen(s). Otherwise, during infusion or aspiration of multi-lumen lines, if one lumen is occluded (or partially occluded), fluid flow would be diverted to the lumen with least resistance, and therefore the caretaker would be unable to access the occluded/partially occluded lumen.

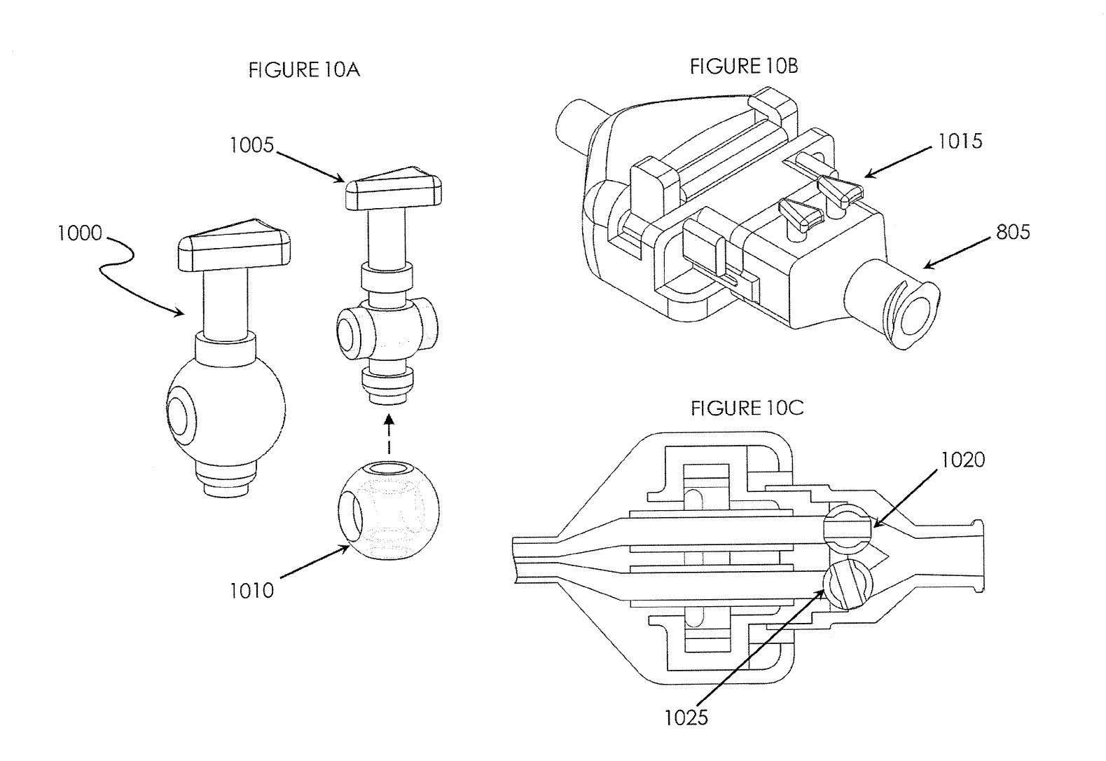

FIGS. 10A-10C show an attachment member with a valve system for use with a single infusion article such as a syringe in conjunction with an embodiment of the present invention. FIG. 10A shows the valve assembly 1000 as a core rigid plastic member 1005 and an elastic member 1010 which is press-fit over the plastic member 1005. FIG. 10B illustrates a perspective view of the catheter hub and the attachment member having a single luer connector 805. A portion of the valve assembly 1015 extends from the attachment member and allows the provider to direct flow to specific lumen(s) according to the flow indicator (e.g., arrow). FIG. 10C shows an overhead cross-sectional view of the catheter hub and attachment member, where the valve assembly 1000 has been turned to restrict flow to the left lumen 1025 and permit flow to the right lumen 1020.

Further, with regards to FIGS. 9A-9B and 10A-10C, the ability of the caretaker to selectively open and/or close a lumen of the attachment member enables proper priming of the device before attachment with the catheter hub.

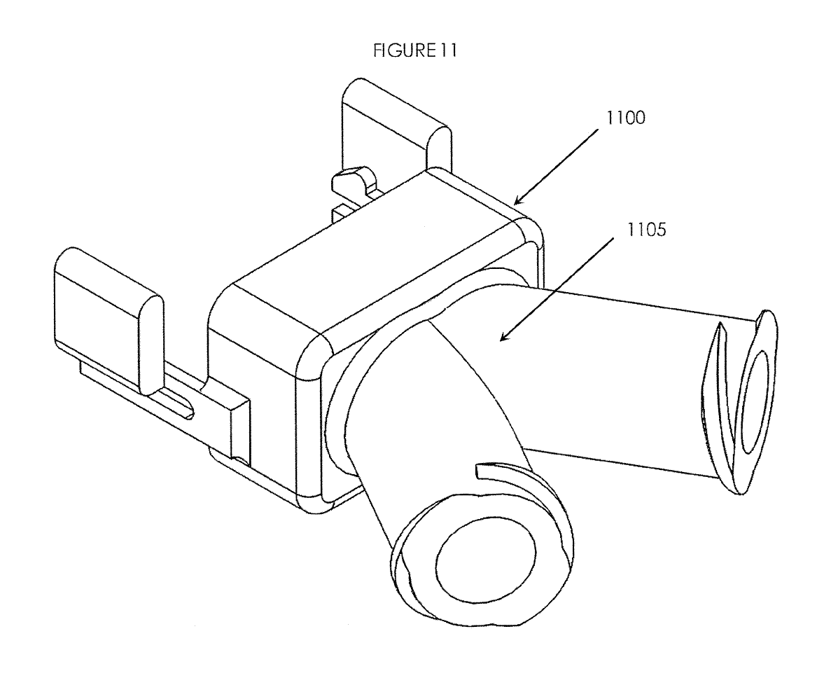

FIG. 11 illustrates a perspective view of an attachment member with individual luer connectors that correspond to the number of lumens of the indwelling catheter, according to an embodiment of the present invention. Each medical connector (e.g., luer) 1105 is neither diverging nor converging within the attachment member 1100, and therefore connects directly to the internal fluid channel of the catheter hub. In this embodiment, the attachment member 1100 acts solely as an adapter between standard medical infusion articles and the catheter hub in an embodiment of the present invention.

As discussed above, the catheter of the claimed invention is capable of providing a connecting hub with close proximity to the catheter insertion site that allows attachment and removal of various extensions, reduces the total surface area of the extravascular portion and the predominant site of a contaminant's origin, facilitates improved placement of insertion-site dressing, and substantially reduces the potential for snagging, irritation, and other problems associated with dangling catheter extensions.

The many features and advantages of the invention are apparent from the detailed specification, and thus, it is intended by the appended claims to cover all such features and advantages of the invention which fall within the true spirit and scope of the invention. Further, since numerous modifications and variations will readily occur to those skilled in the art, it is not desired to limit the invention to the exact construction and operation illustrated and described, and accordingly, all suitable modifications and equivalents may be resorted to, falling within the scope of the invention.

1. A catheter assembly, comprising:
a hub having an outer housing defining a proximal end and a distal end, wherein the distal end of the outer housing defines an opening into an inner space defined by the outer housing, and the proximal end of the housing defines a proximal connector for coupling to an intravenous catheter tube; and
an occluding assembly for blocking a section of a fluid channel disposed between the distal and proximal ends of the hub.

2. The catheter assembly of claim 1, further comprising an attachment member that connects to the distal end of the hub to seal the catheter.

3. The catheter assembly of claim 1, wherein the occluding assembly is a clamping mechanism that includes a clamping member that engages with a clamping support and the clamping support engages with a section of the hub to maintain a position within the hub.

4. The catheter assembly of claim 2, wherein the attachment member connects to a connector region of the hub and is secured by a locking member that engages with a protrusion on the hub.

5. The catheter assembly of claim 1, further comprising an extension tube assembly that is releasably attached to the connector.

6. The catheter assembly of claim 5, wherein the extension tube assembly can be released from the catheter while the catheter unit is affixed to a patient.

7. The catheter assembly of claim 6, wherein the extension tube assembly is releasably engaged to the catheter unit by a mechanical system.

8. The catheter assembly of claim 5, wherein the extension tube assembly includes a locking cap where an extension tube attaches to the hub.

9. The catheter assembly of claim 8, wherein the extension tube comprises a number of extension tubes in a range of 1-4 extension tubes that converge at the locking cap to connect to the hub.

10. The catheter of claim 1, wherein the intravenous catheter tube includes a single lumen or multiple lumens.

11. A catheter assembly, comprising:
a hub having an outer housing defining a proximal end and a distal end, wherein the distal end of the outer housing defines an opening into an inner space defined by the outer housing, and the proximal end of the housing defines a proximal connector for coupling to an intravenous catheter tube; and
an occluding assembly for blocking a section of a fluid channel disposed between the distal and proximal ends of the hub; and
an attachment member that connects to the distal end of the hub to form a fluid-tight seal.

12. The catheter assembly of claim 11, wherein the attachment member includes:
an attachment cap in which fluid connectors are formed; and
a deformable insert that is fitted into the attachment cap.

13. The catheter assembly of claim 12, wherein the insert includes openings that fill a gap between the fluid connectors and a fluid connector region at the distal end of the hub to form the fluid-tight seal.

14. The catheter assembly of claim 11, wherein the attachment member includes:
a female luer connector and a slider configured to divert fluid through lumens of the catheter.

15. The catheter assembly of claim 14, wherein the slider includes an indicator to indicate a position of the slider.

16. The catheter assembly of claim 15, wherein the slider moves laterally within a channel of the attachment member and includes openings and closures.

17. The catheter assembly of claim 16, wherein a fluid lumen proximal to the female luer connector bifurcates into multiple fluid channels.

18. The catheter assembly of claim 11, wherein the attachment member includes:
a female luer connector; and
a valve assembly, a portion of which extends from the attachment member to allow the fluid to be directed to a particular lumen.

19. The catheter assembly of claim 18, wherein the valve assembly is rotated to restrict and permit flow to multiple lumens.

20. The catheter assembly of claim 12, wherein the occluding assembly is a clamping mechanism that includes a clamping member that engages with a clamping support and the clamping support engages with a section of the hub to maintain a position within the hub.

21. A method for releasably attaching a catheter assembly, comprising:
(a) affixing a catheter of claim 1 to a surface of a patient;
(b) engaging the occluding assembly to block fluid movement; and
(c) removing an extension tube assembly that is releasably attached to the connector from the catheter affixed to the patient surface.

22. The method of claim 21, further comprising:
(d) attaching an attachment member to the distal end of the hub to seal the catheter.

23. The method of claim 22, wherein the extension tube assembly is re-attached to the catheter during treatment of the patient.

24. The method of claim 22, wherein the catheter is affixed to the patient surface by suturing or using an adhesive.
